(19) United States
(12) Patent Application Publication
(10) Pub. No.: US 2026/0334814 A1
(43) Pub. Date: Oct. 29, 2026

(54) MASSAGE DEVICE CAPABLE OF MEASURING HEART RATE AND METHOD FOR MEASURING HEART RATE

(71) Applicant: CERAGEM CO., LTD., Cheonan-si, Chungcheongnam-do (KR)

(72) Inventors: Chan Sol Yoon, Hwaseong-si, Gyeonggi-do (KR); Uk Jin Yoon, Seoul (KR); Ki Sung Kim, Cheonan-si, Chungcheongnam-do (KR)

(21) Appl. No.: 19/119,724

(22) PCT Filed: Oct. 11, 2023
(86) PCT No.: PCT/KR2023/015647

§ 371 Date: Apr. 9, 2025

(30) Foreign Application Priority Data
Oct. 11, 2022 (KR) 10-2022-0129658

(51) Int. Cl. A61B 5/11; A61B 5/00; A61H 7/00

(52) U.S. Cl. CPC A61B 5/1102; A61B 5/7207; A61B 5/7225; A61B 5/725; A61H 7/004; A61H 2201/0119; A61H 2201/02; A61H 2230/06

(57) ABSTRACT

Provided is a massage device capable of measuring a heart rate. A massage device capable of measuring a heart rate according to one aspect of the present invention may comprise: a frame placed on the floor; a vibration detection sensor provided on the frame to detect vibration transmitted from the floor to the frame; a massage module including a support part installed on the frame and a massage part provided on the support part to provide a massage to a user's body; a heart rate detection sensor provided in the massage module to detect a heart rate transmitted from the user's body which is seated on the massage part; and a processor which calculates a user's heart rate on the basis of a first signal obtained from the heart rate detection sensor and a second signal obtained from the vibration detection sensor.

### TECHNICAL FIELD

The present invention relates to a massage device capable of measuring a heart rate and a method for measuring a heart rate, and more particularly, to a massage device capable of more accurately measuring a user's heart rate and a method for measuring a heart rate.

### BACKGROUND

In general, heart rate variability (HRV) provides a quantitative indicator for assessing pathological and physiological conditions of the cardiovascular system. Since HRV is influenced by both the sympathetic and parasympathetic nervous systems, it is used as a quantitative indicator of the autonomic nervous system involved in stress-related disorders. Accordingly, products that operate in connection with heartbeats are emerging in health devices, massage devices, and thermotherapeutic devices.

Korean Patent Registration No. 10-2014218 of Ceragem Co., Ltd. discloses a control device of a conventional thermotherapeutic device equipped with a heart rate measurement function. Such a control device is configured to calculate a heart rate from a ballistocardiogram (BCG) signal detected by a weight detection sensor, allowing the thermotherapeutic device to provide an appropriate massage mode to the user based on the calculated heart rate.

Such a control device discloses a configuration for preprocessing a ballistocardiogram (BCG) signal extracted from a weight detection sensor to remove power noise, body weight signal noise, and other interferences. However, such a conventional control device has a problem in that it does not provide a specific technical solution for removing noise generated by vibrations of the floor on which the thermotherapeutic device is placed.

Therefore, there has been a pressing need to develop a massage device that can more accurately calculate the user's heart rate and utilize it by eliminating noise signals caused by vibrations from the floor.
(Patent Document 1) Korean Patent Registration No. 10-2014218

### DISCLOSURE

### Technical Problem

The present invention has been devised in view of the above-described points, and the present invention is directed to providing a massage device capable of accurately measuring a user's heart rate.

The present invention is also directed to providing a massage device capable of displaying a measured heart rate to a user.

The present invention is also directed to providing a massage device capable of providing various massage modes to a user based on a measured heart rate.

The problems of the present invention are not limited to those mentioned above, and other problems not mentioned will be clearly understood by those of ordinary skill in the art from the following description.

### Technical Solution

According to an aspect of the present invention, provided is a massage device capable of measuring heart rate, the massage device including: a frame placed on a floor; a vibration detection sensor provided on the frame and configured to detect vibration transmitted from the floor to the frame; a massage module including a support part installed on the frame and a massage part provided on the support part and configured to provide a massage to a user's body; a heart rate detection sensor provided in the massage module and configured to detect a heart rate transmitted from the user's body seated on the massage part; and a processor configured to determine a user's heart rate based on a first signal obtained from the heart rate detection sensor and a second signal obtained from the vibration detection sensor.

In this case, the first signal may include a heart rate signal containing information on a user's heartbeat and a noise signal other than the heart rate signal, and the processor may be configured to correct the first signal based on the second signal so that the noise signal can be removed.

In this case, the noise signal may include a vibration noise signal caused by vibration transmitted from the floor to the massage module.

In this case, the processor may be configured to determine a correction signal based on the second signal to correct the first signal, and determine the user's heart rate based on the first signal and the correction signal.

In this case, the processor may be configured to determine the correction signal based on the second signal and a correction factor.

In this case, the processor may be configured to adjust the correction factor so that the noise signal of the first signal becomes identical to the correction signal.

In this case, the processor may be configured to use an adaptive filter algorithm to adjust the correction factor.

In this case, the adaptive filter algorithm may include at least one of an LMS (Least Mean Square) filter algorithm, an NLMS (Normalized Least Mean Square) filter algorithm, and an RLS (Recursive Least Square) filter algorithm.

In this case, the processor may be configured to amplify and filter at least one of the first signal and the second signal, and determine the user's heart rate based on the amplified and filtered signal.

In this case, the heart rate detection sensor may include a weight detection sensor configured to detect a weight of a body seated on the massage part.

In this case, the heart rate detection sensor may be disposed on one side of the support part.

In this case, the massage part may include a thermotherapeutic ceramic rotatably coupled to the support part and configured to provide thermotherapy to a user.

In this case, the thermotherapeutic ceramic and the heart rate detection sensor may be each provided in plurality, and the plurality of thermotherapeutic ceramics may include a first thermotherapeutic ceramic and a second thermotherapeutic ceramic arranged along a first line parallel to a user's body length direction, and a third thermotherapeutic ceramic and a fourth thermotherapeutic ceramic arranged along a second line parallel to the first line, and the plurality of heart rate detection sensors may include a first heart rate detection sensor disposed on the first line and a second heart rate detection sensor disposed on the second line.

In this case, the first line and the second line may be symmetrically arranged with respect to the user's body.

According to another aspect of the present invention, provided is a method for measuring a heart rate of a user receiving a massage from a massage device including a frame placed on a floor and a massage module installed on the frame to provide a massage, the method including obtaining a first signal from a heart rate detection sensor provided in the massage module; obtaining a second signal from a vibration detection sensor provided on the frame; and determining the user's heart rate based on the first signal and the second signal.

In this case, the first signal may include a heart rate signal detected based on a user's heartbeat and a noise signal other than the heart rate signal, and the determining the user's heart rate may include correcting the first signal based on the second signal so that the noise signal can be removed.

In this case, the correcting the first signal may include determining a correction signal based on the second signal and a correction factor; and determining the heart rate based on the first signal and the correction signal.

In this case, the correcting the first signal may include adjusting the correction factor so that a noise signal of the first signal becomes identical to the correction signal.

In this case, the adjusting the correction factor may be performed using an adaptive filter algorithm.

In this case, the method may further include amplifying and filtering at least one of the first signal and the second signal.

### Advantageous Effect

The massage device capable of measuring a heart rate and the method for measuring a heart rate according to an example embodiment of the present invention can obtain a first signal from a heart rate detection sensor that detects vibration of a user's body, and can determine the user's heart rate based on the obtained first signal.

In addition, the massage device capable of measuring a heart rate and the method for measuring a heart rate according to an example embodiment of the present invention can obtain a second signal from a vibration detection sensor that detects vibration transmitted from a floor, and can accurately determine the user's heart rate by correcting the first signal based on the obtained second signal.

In addition, the massage device capable of measuring a heart rate and the method for measuring a heart rate according to an example embodiment of the present invention can determine a correction signal based on the second signal and a correction factor, and can more accurately determine the user's heart rate by correcting the first signal based on the determined correction signal.

In addition, the massage device capable of measuring a heart rate and the method for measuring a heart rate according to an example embodiment of the present invention can adjust the correction factor using an adaptive filter algorithm, and can more accurately determine the user's heart rate by correcting the first signal based on the adjusted correction factor to precisely remove a noise signal caused by floor vibration from the first signal.

In addition, the massage device capable of measuring a heart rate according to an example embodiment of the present invention can include a memory for storing data related to the determined heart rate and an interface for displaying the determined heart rate, so that data on the heart rate measured according to the massage can be accumulated and provided to the user.

In addition, the massage device capable of measuring a heart rate according to an example embodiment of the present invention can include a controller configured to determine a massage mode based on the determined heart rate and control the massage module according to the determined massage mode, so that a massage appropriately set according to the heart rate can be provided.

Advantageous effects of embodiments of the present invention are not limited to the above-described effects, and other effects not mentioned will be clearly understood by those of ordinary skill in the art from the present specification and the accompanying drawings.

### BRIEF DESCRIPTION OF THE DRAWINGS

FIG. 1 is a perspective view from above of a massage device capable of measuring heart rate according to an example embodiment of the present invention.

FIG. 2 is a perspective view from below of a massage module and a heart rate detection sensor of the massage device capable of measuring heart rate according to an example embodiment of the present invention.

FIG. 3 is a block diagram schematically illustrating the heart rate detection sensor, vibration detection sensor, processor, memory, interface, and controller of the massage device capable of measuring heart rate according to an example embodiment of the present invention.

FIG. 4 is a block diagram schematically illustrating the processor of the massage device capable of measuring heart rate according to an example embodiment of the present invention.

FIG. 5 is a flowchart of a heart rate measurement method according to an example embodiment of the present invention.

FIG. 6 is a flowchart illustrating in detail the step of calculating heart rate in the heart rate measurement method according to an example embodiment of the present invention.

### MODES OF THE INVENTION

Terms and words used in the present specification and claims should not be construed as limited to their usual or dictionary definition. They should be interpreted as meaning and concepts consistent with the technical idea of the present invention, based on the principle that inventors may appropriately define the terms and concepts to describe their own invention in the best way.

It should be understood that the terms “comprise or include” or “have” or the like when used in this specification, are intended to describe the presence of stated features, numbers, steps, operations, elements, components and/or a combination thereof but not preclude the possibility of the presence or addition of one or more other features, numbers, steps, operations, elements, components, or a combination thereof.

The presence of an element in/on “front”, “rear”. “upper or above or top” or “lower or below or bottom” of another element includes not only being disposed in/on “front”, “rear”, “upper or above or top” or “lower or below or bottom” directly in contact with other elements, but also cases in which another element being disposed in the middle, unless otherwise specified. In addition, unless otherwise specified, that an element is “connected” to another element includes not only direct connection to each other but also indirect connection to each other.

Some embodiments of the present disclosure may be represented by functional block configurations and various processing steps. Some or all of these functional blocks may be implemented in various numbers of hardware and/or software configurations that perform specific functions. For example, the functional blocks of the present disclosure may be implemented by one or more microprocessors, or may be implemented by circuit configurations for certain functions. In addition, for example, the functional blocks of the present disclosure may be implemented in various programming or scripting languages. Functional blocks may be implemented as algorithms running on one or more processors. In addition, the present disclosure may employ conventional technologies for electronic environment setup, signal processing, and/or data processing. Terms such as “mechanism,” “element,” “means,” and “configuration” may be used broadly and are not limited to mechanical and physical components.

The terms “module” or “unit or part” used in the specification refer to software or hardware components, and the “module” or “unit or part” performs certain functions. However, the terms “module” or “unit or part” are not limited to software or hardware. The “module” or “unit or part” may be configured to reside in an addressable storage medium and may be configured to execute one or more processors. Accordingly, as an example, the “module” or “unit or part” may include at least one of software components, object-oriented software components, class components, and task components, as well as processes, functions, attributes, procedures, subroutines, segments of program code, drivers, firmware, microcode, circuits, data, databases, data structures, tables, arrays, or variables. The components and the “modules” or “units or parts” may be combined into a smaller number of components and “modules” or “units or parts” or further separated into additional components and “modules” or “units or parts,” depending on the functions provided therein.

According to an embodiment of the present disclosure, the “module” or “unit or part” may be implemented by a processor and a memory. The “processor” should be broadly interpreted to include a general-purpose processor, a central processing unit (CPU), a microprocessor, a digital signal processor (DSP), a controller, a microcontroller, and a state machine. In some environments, the “processor” may also refer to an application-specific integrated circuit (ASIC), a programmable logic device (PLD), or a field-programmable gate array (FPGA). For example, the “processor” may also refer to a combination of processing devices, such as a combination of a DSP and a microprocessor, a combination of multiple microprocessors, a combination of one or more microprocessors coupled with a DSP core, or any other such components. In addition, the “memory” should be broadly interpreted to include any electronic component capable of storing electronic information. The “memory” may also refer to various types of processor-readable media, such as random access memory (RAM), read-only memory (ROM), non-volatile random access memory (NVRAM), programmable read-only memory (PROM), erasable programmable read-only memory (EPROM), electrically erasable PROM (EEPROM), flash memory, magnetic or optical data storage devices, and registers. If the processor can read information from and/or write information to the memory, the memory is said to be in electronic communication with the processor. Memory integrated into the processor is in electronic communication with the processor.

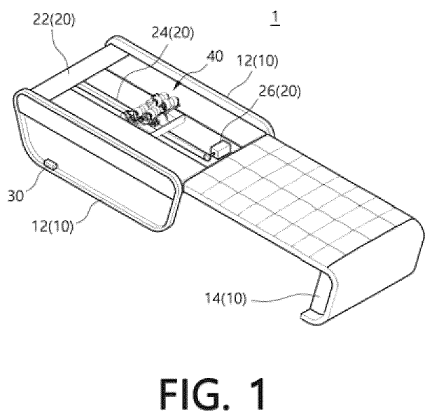

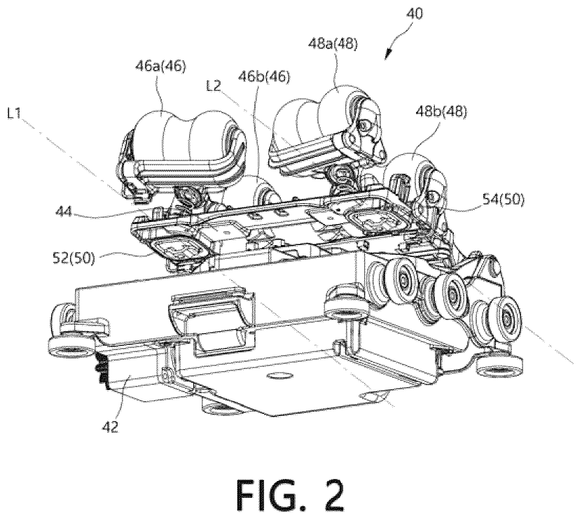

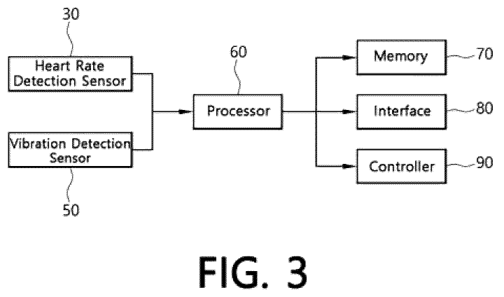

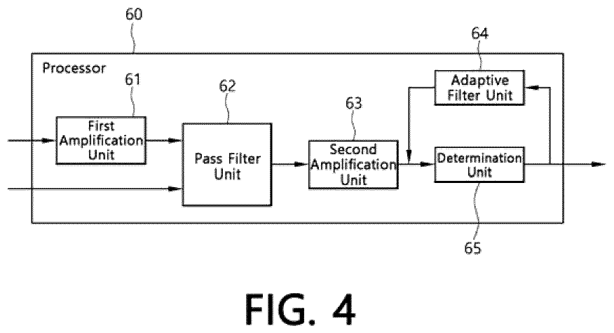

FIG. 1 is a perspective view from above of a massage device capable of measuring heart rate according to an example embodiment of the present invention. FIG. 2 is a perspective view from below of a massage module and a heart rate detection sensor of the massage device capable of measuring heart rate according to an example embodiment of the present invention. FIG. 3 is a block diagram schematically illustrating the heart rate detection sensor, vibration detection sensor, processor, memory, interface, and controller of the massage device capable of measuring heart rate according to an example embodiment of the present invention. FIG. 4 is a block diagram schematically illustrating the processor of the massage device capable of measuring heart rate according to an example embodiment of the present invention.

Referring to FIGS. 1 and 2, a massage device 1 capable of measuring heart rate according to an example embodiment of the present invention may include lower and upper frames 10 and 20, a vibration detection sensor 30, a massage module 40, and a heart rate detection sensor 50.

As shown in FIG. 1, the lower and upper frames 10 and 20 are configured to allow a user to lie down or place a part of their body thereon, and the lower and upper frames 10 and 20 may be placed on a floor surface.

In addition, the lower and upper frames 10 and 20 may provide a base on which the massage module 40 is installed. The massage module 40, provided on the upper side of the lower and upper frames 10 and 20, provides a massage to the user's body positioned on the lower frame 10 and the upper frame 20.

The massage module 40 is provided with a heart rate detection sensor 50, which can detect heartbeats transmitted from the user's body and convert them into electrical signals. Hereinafter, the above signal is referred to as a first signal.

The lower frame 10 is provided with a vibration detection sensor 30, which can detect vibrations transmitted from the floor and convert them into electrical signals. Hereinafter, the above signal is referred to as a second signal.

Referring also to FIG. 2, the processor 60 determines the heart rate based on the first and second signals. That is, the massage device 1 capable of measuring heart rate according to an example embodiment of the present invention may determine a heart rate more accurately based on signals acquired from a plurality of sensors and may use the determined heart rate for controlling the massage module 40 or provide it to the user.

Hereinafter, each component of the massage device capable of measuring heart rate according to an example embodiment of the present invention will be described in more detail.

Referring to FIG. 1, the lower and upper frames 10 and 20 of the massage device 1 capable of measuring heart rate according to an example embodiment of the present invention may include a lower frame 10 supported by a floor surface and an upper frame 20 provided above the lower frame 10, on which at least a portion of the user's body is placed. The lower and upper frames 10 and 20 may be made of a material with high rigidity, such as metal or reinforced plastic, to ensure sufficient supporting strength.

In the illustrated embodiment, the lower frame 10 includes a first lower frame 12 for supporting the upper frame 20 on which the upper body of a user is placed and a second lower frame 14 for supporting the lower body of the user.

The first lower frame 12 is formed as a pair, and the pair of first lower frames 12 may be spaced apart from each other by a predetermined distance in the left-right direction. The upper frame 20 described above may be provided between the pair of first lower frames 12.

The upper frame 20 is configured to support the user's upper body with the supporting force provided by the lower frame 10 and to provide a base for installing the massage module 40, which can provide a massage to the user.

The upper frame 20 may have a structure to assist the function of the massage module 40. In the illustrated embodiment, the massage module 40 is movably coupled to the upper frame 20 so as to move along the user's body length direction and provide a massage.

To this end, in the present embodiment, the upper frame 20 includes a base plate 22 supported by the first lower frame 12 and a rail 24 formed along the user's body length direction on the upper portion of the base plate 22. In addition, an actuator 26 is provided on one side of the base plate 22 to supply driving force for the movement of the massage module 40.

However, the shape and structure of the lower and upper frames 10 and 20 of the massage device 1 capable of measuring heart rate according to an example embodiment of the present invention are not limited to the illustrated embodiment, and various modifications may be made to the shape and structure of the lower and upper frames 10 and 20 without departing from the spirit of the present invention.

For example, the shape and structure of the lower and upper frames 10 and 20 may be appropriately configured according to the characteristics of the space in which the massage device 1 is placed, the characteristics of the body part to which the massage is provided, and the functions of the massage module 40.

Meanwhile, referring to FIG. 1, the lower frame 10 may be provided with a vibration detection sensor 30 that detects vibrations transmitted to the lower and upper frames 10 and 20 from the floor surface on which the massage device 1 is placed and converts the detected vibrations into the aforementioned second signal.

The vibration detection sensor 30 may be a variable-resistance-type vibration detection sensor, a light-quantity-detection-type vibration detection sensor, or a piezoelectric vibration detection sensor. However, the type of the vibration detection sensor is not particularly limited as long as it can detect vibrations transmitted from the floor surface to the lower and upper frames 10 and 20.

In the illustrated embodiment, the vibration detection sensor 30 is installed on an outer portion of the first lower frame 12. However, the installation position of the vibration detection sensor is not particularly limited as long as it can detect vibrations transmitted from the floor surface to the lower and upper frames 10 and 20.

For example, the vibration detection sensor 30 may be mounted inside the first lower frame 12, installed on the second lower frame 14, or installed on the base plate 22.

Referring to FIGS. 1 and 2, as described above, a massage module 40 is provided on the upper side of the lower and upper frames 10 and 20. In the present embodiment, the massage module 40 includes a transfer part 42, a support part 44, and massage parts 46 and 48.

The transfer part 42 is a box-shaped structure configured to move the massage parts 46 and 48 along the user's body length direction. To this end, rollers may be provided on a side or bottom of the transfer part 42 to be movably coupled to the rail 24 on the base plate 22.

In addition, the transfer part 42 may be connected to the aforementioned actuator 26 in a predetermined manner to receive a driving force. Accordingly, the transfer part 42 can receive the driving force and move the support part 44 and the massage parts 46 and 48, which will be described later.

The support part 44 may be fixedly coupled to an upper side of the transfer part 42. The support part 44 is a plate-shaped member extending in the horizontal direction and is configured to support the massage parts 46 and 48, which will be described later.

In the present embodiment, the massage parts 46 and 48 may include a first massage part 46 and a second massage part 48, which can provide massages to different parts of the body, as illustrated.

The first and second massage parts 46 and 48 may include a plurality of thermotherapeutic ceramics 46 and 48, as illustrated. In this case, the plurality of thermotherapeutic ceramics 46 and 48 may be rotatably coupled to the support part 44.

The first massage part 46 may include a first thermotherapeutic ceramic 46a and a second thermotherapeutic ceramic 46b, which are arranged along a first line L1 parallel to the user's body length direction, and the second massage part 48 may include a third thermotherapeutic ceramic 48a and a fourth thermotherapeutic ceramic 48b, which are arranged along a second line L2 that is parallel to the first line L1 but spaced apart by a predetermined distance.

In this case, the first line L1 and the second line L2 may be symmetrically arranged with respect to the user's body. Accordingly, the first massage part 46 and the second massage part 48 can symmetrically support the user's body and provide a massage.

Through this, the load from the user's body can be evenly distributed, thereby increasing the structural stability of the massage module 40 and effectively providing a massage that is suitable for the body's structure.

Meanwhile, in the illustrated embodiment, the massage parts 46 and 48 are made of thermotherapeutic ceramics as mentioned above, however, this is not limiting, and the massage parts 46 and 48 may be composed of various configurations that move in a predetermined manner to provide a massage to the user.

For example, the massage parts 46 and 48 may include a massage protrusion that moves up and down to apply localized pressure to the user's body, or a massage roller that moves in a rolling motion to apply gentle pressure to the user's body.

At this point, referring back to FIG. 2, the massage module 40 may be equipped with a heart rate detection sensor 50. As described above, the heart rate detection sensor 50 is configured to detect vibrations of the user's body that contain information related to the heart rate and convert them into a first signal, which is an electrical signal.

The heart rate detection sensor 50 may include various types of sensors capable of detecting body vibrations. In the present embodiment, the heart rate detection sensor 50 is a weight detection sensor comprising a load cell capable of detecting the user's body weight.

The weight detection sensor may detect a body weight signal that includes minute ballistocardiogram signals. In this case, the ballistocardiogram (BCG) signal refers to a signal obtained by detecting body vibrations accompanying heartbeats.

In this case, the heart rate detection sensor 50 may be provided on one side of the support part 44 that supports the massage parts 46 and 48, specifically on the lower side of the support part 44 with reference to FIG. 2. Through this, the heart rate detection sensor 50 can stably and accurately detect the users body vibrations while minimizing the influence of the movement of the massage parts 46 and 48.

In addition, a plurality of heart rate detection sensors 50 may be provided. In the illustrated embodiment, the plurality of heart rate detection sensors 50 include a first heart rate detection sensor 52 disposed along a first line L1 and a second heart rate detection sensor 54 disposed along a second line L2. Accordingly, the first and second heart rate detection sensors 52 and 54 can stably and accurately detect body vibrations based on the evenly distributed body weight.

However, the location where the heart rate detection sensor 50 is installed in the massage module 40 is not limited to the support part 44 on the first or second line L1 or L2 as illustrated. As long as it can detect the user's body vibrations and measure the heart rate, the location is not specifically restricted. In other words, if the heart rate detection sensor 50 can stably and accurately measure the heart rate, it may be installed on the massage parts 46 and 48, which operate in a predetermined manner and directly apply pressure to the user's body.

Meanwhile, the first signal obtained from the heart rate detection sensor 50 may consist of a heart rate signal (i.e., a ballistocardiogram signal) containing information about the user's heart rate, as well as noise signals other than the heart rate signal.

The noise signals interfere with accurately determining the heart rate from the first signal. The noise signals may include signals caused by vibrations transmitted from the floor through the frame to the massage module 40.

In the present embodiment, in order to minimize the influence of the aforementioned noise signals, the aforementioned vibration detection sensor 30 (illustrated in FIG. 1) is introduced. The process of accurately determining the heart rate by considering the noise signals will be described later, along with the method for determining the heart rate according to an example embodiment of the present invention.

Referring to FIGS. 1 and 3, a massage device 1 capable of measuring heart rate according to an example embodiment of the present invention may further include a processor 60, a memory 70, an interface 80, and a controller 90, which can be used to measure the heart rate. The processor 60, memory 70, interface 80, and controller 90 may be installed on one side of the lower and upper frames 10 and 20, or may be mounted inside the lower and upper frames 10 and 20.

The processor 60 is configured to receive the first signal from the heart rate detection sensor 50 and determine the user's heart rate based on the first signal.

The processor 60 may be implemented as hardware, software, and/or a combination thereof. For example, the processor 60 may be implemented using an electrical circuit processed by hardware, or implemented by a processor, central processing unit (CPU), controller, arithmetic logic unit, computational logic circuit, digital signal processing unit, microcomputer, FPGA, system on chip (SoC), programmable logic unit, microprocessor or any device capable of performing the functions described below.

In addition, the processor 60 may be configured to process instructions from a computer program by performing basic arithmetic, logic, and input/output operations. The instructions may be provided to the processor 60 by the memory 70 or the like, as described later. For example, the processor 60 may be configured to execute instructions received according to program code stored in a recording device such as the memory 70.

Referring to FIG. 3 and FIG. 4, the processor 60 may receive a second signal from the vibration detection sensor 30 and, based on the received second signal, correct the first signal in order to accurately determine the heart rate from the first signal. To this end, the processor 60 may include a first amplification unit 61, a pass filter unit 62, a second amplification unit 63, an adaptive filter unit 64, and a determination unit 65.

As such, the massage device capable of measuring heart rate according to an example embodiment of the present invention can accurately determine the user's heart rate by correcting the first signal, which contains a mixture of a heart rate signal carrying information about heartbeat and a noise signal caused by vibrations from the floor surface, based on the second signal obtained by detecting the vibrations of the floor surface.

The specific functions of the processor 60 and components constituting the processor 60, as well as a method for determining a heart rate based on the first signal and the second signal, will be described below together with a method for measuring a heart rate according to an example embodiment of the present invention.

Referring to FIG. 3, the processor 60 may transmit the data related to the determined heart rate to the memory 70. The memory 70 may store the received data. In addition, the memory 70 may provide the stored data to other components of the massage device as needed. For example, the memory 70 may transmit the data related to the heart rate to the interface 80 and/or the controller 90, which will be described later.

The memory 70 may include any non-transitory computer-readable recording medium. According to an example embodiment, the memory 70 may include non-volatile large-capacity storage devices such as ROM, SSD, flash memory, disk drives, as well as RAM (random access memory), ROM (read-only memory), disk drives, SSD (solid state drive), flash memory, and the like. In addition, as described above, at least one program code (e.g., code for determining the heart rate based on the first signal and the second signal) may be stored in the memory 70. Such software components may be loaded from a computer-readable recording medium separate from the memory 70. Such a separate computer-readable recording medium may include a recording medium that can be directly connected to the processor 60 of the present embodiment, for example, a computer-readable recording medium such as a floppy drive, disk, tape, DVD/CD-ROM drive, or memory card. As another example, the software components may be delivered from an external source via a separate communication module, rather than a computer-readable recording medium, and loaded into the memory 70.

Meanwhile, referring back to FIG. 3, the processor 60 may transmit the data related to the determined heart rate to the interface 80. The interface 80 is configured to interact with a user, transmit information, or receive input signals. In the present embodiment, the user may be provided with data on the determined heart rate through the interface 80.

To this end, the interface 80 may be configured to input information and/or data into the processor 60 or the memory 70, or to output information and/or data generated from the processor 60 or the memory 70. The interface 80, as an example of an input/output means, may include a button member, a digital signal indicator, or a display equipped with a touch screen.

In addition, the processor 60 may transmit the determined heart rate data to the controller 90.

The controller 90 is configured to determine a massage mode based on the heart rate data determined by the processor 60 and to control the massage parts 46 and 48 (shown in FIG. 2) and the actuator 26 (shown in FIG. 1) to perform the determined massage mode. Of course, the controller 90 may also receive the heart rate data determined by the processor 60 from the memory 70.

The controller 9X) may be implemented using an electrical circuit processed by hardware, or implemented by a processor, central processing unit (CPU), controller, arithmetic logic unit, computational logic circuit, digital signal processing unit, microcomputer, FPGA, system on chip (SoC), programmable logic unit, microprocessor or any device capable of performing the functions described below.

In this case, the controller 90 may determine a massage mode based on the determined heart rate. In addition, the controller 90 may automatically set a massage mode capable of alleviating the user's condition by determining the user's condition based on the heart rate determined by the processor 60.

For example, the controller 90 may control the corresponding massage mode to be actively performed by automatically setting a massage pattern, massage intensity, and massage temperature of the massage mode to activate the sympathetic nervous system or the parasympathetic nervous system.

For example, when the determined heart rate exceeds a reference heart rate according to the input age and gender of the user, the controller 90 may control a parasympathetic promotion mode to be performed to activate the parasympathetic nervous system of the user in order to stabilize the body rhythm. In this case, the reference heart rate may be determined based on data input by the user through the interface 80 and data on resting heart rates corresponding to age and gender.

In addition, when the determined heart rate is less than the reference heart rate according to the input age and gender of the user, the controller 90 may control a sympathetic promotion mode to be performed to activate the sympathetic nervous system of the user in order to stimulate the body rhythm.

In addition, when the determined heart rate is equal to the reference heart rate according to the input age and gender of the user, the controller 90 may control a standard mode in which the massage mode operates in a previously set state to be performed.

Here, a massage temperature, massage pattern, and massage intensity for each execution mode may be set according to a state-specific massage mode stored in the memory 70. In addition, a massage temperature, massage pattern, and massage intensity for each execution mode may also be directly set by the user through the interface 80.

As described above, according to the present embodiment, the processor 60 accurately determines the heart rate, and the controller 90 determines a massage mode based on the determined heart rate and controls the massage part and actuator, thereby effectively providing a massage mode more suitable for the users condition.

Meanwhile, although the controller 90 is illustrated in FIG. 3 as a component configured separately from the processor 60, it is not limited thereto, and the controller 90 may be configured to be included in the processor 60.

Hereinafter, a method for measuring a heart rate according to an example embodiment of the present invention will be described together with a detailed configuration of the processor described above.

To aid in the understanding of the invention, the present disclosure will describe a method for measuring a heart rate by using the massage device capable of measuring a heart rate according to the above-described embodiment of the present invention.

However, the method for measuring a heart rate according to an example embodiment of the present invention may be applied not only to the massage device capable of measuring a heart rate as illustrated in FIGS. 1 to 4, but also to other massage devices that include a heart rate detection sensor for detecting a user's heartbeat and a vibration detection sensor for detecting vibrations of a floor surface.

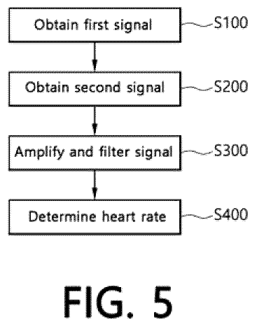

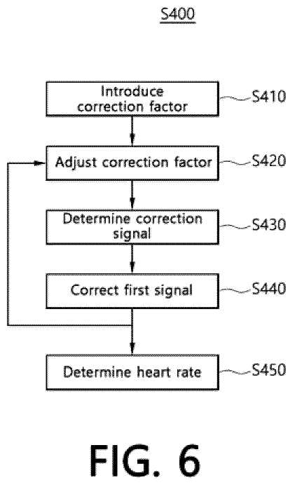

FIG. 5 is a flowchart of a heart rate measurement method according to an example embodiment of the present invention. FIG. 6 is a flowchart illustrating in detail the step of calculating heart rate in the heart rate measurement method according to an example embodiment of the present invention.

Referring to FIG. 5 together with FIGS. 1 and 2, in the method for measuring a heart rate according to an example embodiment of the present invention, the heart rate detection sensor 50 provided in the massage module 40 acquires a first signal (S100), and the vibration detection sensor 30 provided in the lower frame 10 acquires a second signal (S200).

At this time, the order of the step of acquiring the first signal (S100) and the step of acquiring the second signal (S200) is not particularly limited. However, as will be described later, since the second signal is used to correct the first signal, it is preferable that the first signal and the second signal be acquired simultaneously.

Referring to FIG. 5 together with FIGS. 3 and 4, in the method for measuring a heart rate according to an example embodiment of the present invention, after the first and second signals are acquired (S100, S200), at least one of the first and second signals is amplified and filtered (S300).

More specifically, in the step of amplifying and filtering the signals (S300) according to the present embodiment, the first amplification unit 61 primarily amplifies the first signal, the pass filter unit 62 filters the primarily amplified first signal, and the second amplification unit 63 secondarily amplifies the filtered first signal.

In addition, in the step of amplifying and filtering the signals (S300) according to the present embodiment, the pass filter unit 62 filters the second signal, and the second amplification unit 63 amplifies the filtered second signal.

At this time, the first amplification unit 61 may be implemented as a preamplifier, the pass filter unit 62 may be implemented as a low-pass filter and a high-pass filter, and the second amplification unit 63 may be implemented as a main amplifier.

Accordingly, the first and second signals may be preprocessed to facilitate processing in a subsequent step and to accurately determine the heart rate. Meanwhile, the process in which the first and second signals are amplified and filtered is not limited to the above-described process and may be repeatedly performed in various orders.

Referring to FIG. 5 together with FIGS. 3 and 4, in the method for measuring a heart rate according to an example embodiment of the present invention, the processor 60 amplifies and filters the first and second signals (S300), and then determines the heart rate (S400).

More specifically, referring to FIG. 6, in the step of determining the heart rate (S400) according to the present embodiment, the determination unit 65 of the processor 60 introduces a correction factor (S410) and determines a correction signal based on the second signal and the correction factor (S430). The step of modifying the correction factor (S420) illustrated in FIG. 6 will be described later together with the adaptive filter unit 64 illustrated in FIG. 4.

At this time, the correction factor and the correction signal may be defined in various ways to effectively correct the first signal based on the second signal. For example, the correction signal may be defined as a product of the second signal and the correction factor according to the following [Equation (1)].

$${y}_{j} = {x}_{j}^{T} {ω}_{j} = {ω}_{j}^{T} {x}_{j} \qquad [ Equation ( 1 ) ]$$

Where, in [Equation (1)], yⱼ denotes the correction signal, xⱼ denotes the second signal, and ωⱼ denotes the correction factor.

Referring again to FIG. 6 together with FIG. 4, in the step of determining the heart rate (S400) according to the present embodiment, the determination unit 65 of the processor 60 determines the correction signal (S430), and then corrects the first signal based on the correction signal (S440).

As described above, the first signal may include a heart rate signal detected based on the user's heartbeat and a noise signal other than the heart rate signal, particularly a noise signal caused by vibrations of the floor surface.

At this time, in the step of correcting the first signal (S440), the first signal may be corrected in various ways so that the above-described noise signal is effectively removed. For example, the first signal may be corrected by subtracting the correction signal from the first signal according to the following [Equation (2)].

$${e}_{j} = {d}_{j} - {y}_{j} = {d}_{j} - {x}_{j}^{T} {ω}_{j} = {d}_{j} = - {ω}_{j}^{T} {x}_{j} \qquad [ Equation ( 2 ) ]$$

Where, in [Equation (2)], eⱼ denotes the corrected first signal, and dⱼ denotes the first signal. Hereinafter, the corrected first signal is referred to as an output signal.

Referring again to FIG. 6 together with FIG. 4, in the step of determining the heart rate (S400) according to the present embodiment, the first signal is corrected to generate an output signal (S440), and the heart rate is then determined based on the generated output signal (S450).

At this time, the step of determining the heart rate based on the output signal (S450) may be performed in various ways. For example, the determination unit 65 of the processor 60 may detect a peak signal based on the output signal. At this time, the peak signal is a signal corresponding to the heart rate signal. Thereafter, the determination unit 65 may determine the heart rate based on the number of peak points of the heart rate signal detected per unit time.

As such, according to the method for determining a heart rate of the present embodiment, the first signal is corrected based on the second signal acquired by detecting vibrations of the floor surface, so that noise signals caused by the vibrations of the floor surface can be selectively removed from the first signal, thereby enabling accurate measurement of the heart rate.

Meanwhile, referring again to FIG. 6 together with FIG. 4, in the step of determining the heart rate (S400) according to an embodiment of the present invention, in order to more accurately measure the heart rate, a correction factor may be introduced (S410) and then the correction factor may be adjusted (S420).

To this end, in the method for determining a heart rate according to the present embodiment, the adaptive filter unit 64 determines an adjusted correction factor by using the correction factor before adjustment and the output signal determined based on the correction factor before adjustment.

In the following equation, ωⱼ denotes the correction factor before adjustment, and wⱼ₊₁ denotes the adjusted correction factor. At this time, the adjusted correction factor may be adjusted so that a correction signal, which is obtained by multiplying the second signal by the adjusted correction factor, becomes identical to the noise signal caused by vibrations of the floor surface included in the first signal.

To this end, in the step of modifying the correction factor (S420) according to the present embodiment, the adaptive filter unit 64 determines a squared error ((eⱼ)²) according to the following [Equation (3)].

$$( {{e}_{j} )}^{2} = {d}_{j}^{2} - 2 {d}_{j} {x}_{j}^{T} {ω}_{j} + {ω}_{j}^{T} {x}_{j} {x}_{j}^{T} {ω}_{j} \qquad [ Equation ( 3 ) ]$$

Thereafter, in the step of modifying the correction factor (S420) according to the present embodiment, the adaptive filter unit 64 determines a mean squared error ( [symbol] ) according to the following [Equation (4)]. At this time, E[ ] denotes an expectation operator.

$${\text{?}}_{j}^{2} = E [ {e}_{j}^{2} ] = E [ {d}_{j}^{2} ] - 2 [ {d}_{j} {x}_{j}^{T} ] {ω}_{j} + {ω}_{j}^{T} E [ {x}_{j} {x}_{j}^{T} ] {ω}_{j} \qquad [ Equation ( 4 ) ]$$

At this time, the mean squared error may be expressed as shown in the following [Equation (5)]. At this time,

[symbol]

$$E [ {d}_{j}^{2} ] - 2 {p}^{T} ω + {ω}^{T} R ω \qquad [ Equation ( 5 ) ]$$

Thereafter, in the step of modifying the correction factor (S420) according to the resent embodiment, the adaptive filter unit 64 determines a gradient of the mean squared error

$$( \frac{\partial {\text{?}}_{j}^{2}}{\partial ω} )$$

according to the following [Equation (6)].

$$\frac{\partial {\text{?}}_{j}^{2}}{\partial ω} = \frac{\partial }{\partial ω} ( E [ {d}_{j}^{2} ] - 2 {p}^{T} ω + {ω}^{T} R ω ) \qquad [ Equation ( 6 ) ]$$

At this time, the gradient of the mean squared error

$$( \frac{\partial {\text{?}}_{j}^{2}}{\partial ω} or ∇ )$$

may be expressed as shown in the following [Equation (7)].

∇=−2p+2Rω [Equation (7)]

Thereafter, in the step of modifying the correction factor (S420) according to the present embodiment, the adaptive filter unit 64 determines an adjusted correction factor that makes the gradient of the mean squared error zero. At this time, the adjusted correction factor may be expressed as shown in the following [Equation (8)] according to the Wiener-Hopf equation.

ωⱼ₊₁=R¹ω [Equation (8)]

The adjusted correction factor may be determined through the above-described process. At this time, the step of modifying the correction factor (S420) may be repeatedly performed while the heart rate is being determined, so that the correction factor can be continuously updated.

Referring again to FIG. 6 together with FIG. 4, in the step of determining the heart rate (S400) according to the present embodiment, after the adaptive filter unit 64 of the processor 60 adjusts the correction factor (S420), the determination unit 65 of the processor 60 determines a correction signal based on the adjusted correction factor (S430), corrects the first signal based on the determined correction signal to generate an output signal (S440), and determines the heart rate based on the output signal (S450).

Accordingly, according to the method for determining a heart rate of the present embodiment of the present invention, the correction factor is adjusted so that the correction signal becomes identical to the noise signal, whereby the noise signal caused by vibrations of the floor surface can be selectively and accurately removed from the first signal, enabling the heart rate to be determined with higher accuracy.

Meanwhile, referring again to FIG. 6 together with FIG. 4, in the step of modifying the correction factor (S420), the correction factor may be adjusted using an estimated gradient ( [symbol] ). At this time, the estimated gradient may be defined as a derivative of the squared error, as shown in the following [Equation (9)].

$$\hat{∇} = \frac{\partial {e}_{j}^{2}}{\partial ω} \qquad [ Equation ( 9 ) ]$$

That is, the adaptive filter unit 64 may determine an adjusted correction factor using the following [Equation (10)], which is derived based on the estimated gradient and the steepest descent method.

ωⱼ₊₁=ωⱼ+μ(− [symbol] _{J}) [Equation (10)]

At this time, in [Equation (10)], μ denotes a step size. If the step size is large, the correction factor that is iteratively updated may converge quickly to a predetermined value, but it may also increase the error in the steady state or diverge without finding a solution.

Conversely, if the step size is small, the correction factor that is iteratively updated may converge slowly to a predetermined value, but the error in the steady state can be minimized. To this end, the step size may be appropriately adjusted. For example, the step size may be set to a value ranging from 0.1 to 1.

Thereafter, the adaptive filter unit 64 may calculate the estimated gradient using the following [Equation (11)].

$$\hat{∇} = \frac{\partial {e}_{j}^{2}}{\partial ω} = 2 {e}_{j} \frac{\partial {e}_{j}}{\partial ω} = 2 {e}_{j} \frac{\partial ( {d}_{j} - {x}_{j}^{T} ω )}{\partial ω} = - 2 {e}_{j} {x}_{j} \qquad [ Equation ( 11 ) ]$$

Thereafter, the adaptive filter unit 64 may derive the following [Equation (12)] by combining [Equation (10)] and [Equation (11)], and may determine an adjusted correction factor based on the derived [Equation (12)].

ωⱼ₊₁=ωⱼ+2μeⱼxⱼ [Equation (12)]

The adjusted correction factor determined through the above-described process may be used to more accurately determine the heart rate.

Meanwhile, referring again to FIG. 6 together with FIG. 4, the step of modifying the correction factor (S420) described above may be performed by the adaptive filter unit 64 of the processor 60 using an adaptive filter algorithm.

In this case, as the adaptive filter algorithm, at least one of an LMS (Least Mean Square) filter algorithm, an NLMS (Normalized Least Mean Square) filter algorithm, and an RLS (Recursive Least Square) filter algorithm may be used.

Preferably, an NLMS (Normalized Least Mean Square) filter algorithm may be used, which is less sensitive to distribution changes of the input signal and is suitable for an unknown non-stationary environment, and which can overcome the problem of amplifying gradient noise caused by the weight vector being directly proportional to the power of the tap input.

Meanwhile, the above-described method for measuring a heart rate according to an example embodiment of the present invention may be provided as a computer program stored in a computer-readable recording medium for execution by a computer. The medium may either permanently store a computer-executable program or temporarily store the program for execution or download. In addition, the medium may be various recording or storage means in the form of a single hardware or a combination of multiple hardware devices, and is not limited to media directly connected to a computer system, but may also be distributed across a network.

Examples of the medium may include magnetic media such as hard disks, floppy disks, and magnetic tapes; optical recording media such as CD-ROMs and DVDs; magneto-optical media such as floptical disks; and ROM, RAM, flash memory, and the like, which are configured to store program instructions. In addition, other examples of the medium may include recording media or storage media managed by app stores that distribute applications, or by various sites and servers that supply or distribute other types of software.

The methods, operations, or techniques of the present disclosure may also be implemented by various means. For example, such techniques may be implemented in hardware, firmware, software, or a combination thereof. Those skilled in the art will understand that the various example logical blocks, modules, circuits, and algorithm steps described in connection with the present disclosure may be implemented in electronic hardware, computer software, or combinations of both.

To clearly illustrate this interchangeability of hardware and software, various example components, blocks, modules, circuits, and steps have been generally described above in terms of their functionality. Whether such functionality is implemented as hardware or as software depends on design requirements imposed on the particular application and the overall system. Those skilled in the art may implement the described functionality in various ways for each particular application; however, such implementations should not be construed as departing from the scope of the present disclosure.

In a hardware implementation, processing units used to perform the techniques may be implemented within one or more ASICs, DSPs, digital signal processing devices (DSPDs), programmable logic devices (PLDs), field programmable gate arrays (FPGAs), processors, controllers, microcontrollers, microprocessors, electronic devices, other electronic units designed to perform the functions described in the present disclosure, computers, or combinations thereof.

Therefore, the various example logical blocks, modules, and circuits described in connection with the present disclosure may be implemented or performed using a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but alternatively, the processor may be any conventional processor, controller, microcontroller, or state machine. The processor may also be implemented as a combination of computing devices, such as a DSP and a microprocessor, multiple microprocessors, one or more microprocessors in conjunction with a DSP core, or any other configuration of such combinations.

In a firmware and/or software implementation, the techniques may be implemented as instructions stored on a computer-readable medium such as RAM (random access memory), ROM (read-only memory). NVRAM (non-volatile random access memory), PROM (programmable read-only memory), EPROM (erasable programmable read-only memory), EEPROM (electrically erasable PROM), flash memory, compact disc (CD), magnetic or optical data storage devices, and the like. The instructions may be executable by one or more processors and may cause the processor(s) to perform certain aspects of the functionality described in the present disclosure.

Although the above-described embodiments have been described as utilizing aspects of the present disclosure in one or more standalone computer systems, the present disclosure is not limited thereto and may be implemented in connection with any computing environment, such as a network or distributed computing environment. Furthermore, aspects of the subject matter of the present disclosure may be implemented across multiple processing chips or devices, and storage may likewise be affected across multiple devices. Such devices may include PCs, network servers, and portable devices.

Although example embodiments of the present invention have been described above, the scope of the present invention is not limited to the embodiments set forth herein. Those skilled in the art who understand the spirit of the present invention may readily propose other embodiments by adding, modifying, deleting, or supplementing components within the scope of the present invention, and such embodiments should also be regarded as falling within the scope of the present invention.

1. A massage device capable of measuring heart rate, the massage device comprising:
a frame placed on a floor;
a vibration detection sensor provided on the frame and configured to detect vibration transmitted from the floor to the frame;
a massage module including a support part installed on the frame and a massage part provided on the support part and configured to provide a massage to a user's body;
a heart rate detection sensor provided in the massage module and configured to detect a heart rate transmitted from the user's body seated on the massage part; and
a processor configured to determine a user's heart rate based on a first signal obtained from the heart rate detection sensor and a second signal obtained from the vibration detection sensor.

2. The massage device of claim 1,
wherein the first signal comprises a heart rate signal containing information on a user's heartbeat and a noise signal other than the heart rate signal, and
wherein the processor is configured to correct the first signal based on the second signal so that the noise signal can be removed.

3. The massage device of claim 2, wherein the noise signal comprises a vibration noise signal caused by vibration transmitted from the floor to the massage module.

4. The massage device of claim 2,
wherein the processor is configured to:
determine a correction signal based on the second signal to correct the first signal, and
determine the user's heart rate based on the first signal and the correction signal.

5. The massage device of claim 4, wherein the processor is configured to determine the correction signal based on the second signal and a correction factor.

6. The massage device of claim 5, wherein the processor is configured to adjust the correction factor so that the noise signal of the first signal becomes identical to the correction signal.

7. The massage device of claim 6, wherein the processor is configured to use an adaptive filter algorithm to adjust the correction factor.

8. The massage device of claim 7, wherein the adaptive filter algorithm comprises at least one of an LMS (Least Mean Square) filter algorithm, an NLMS (Normalized Least Mean Square) filter algorithm, and an RLS (Recursive Least Square) filter algorithm.

9. The massage device of claim 1, wherein the processor is configured to amplify and filter at least one of the first signal and the second signal, and determine the user's heart rate based on the amplified and filtered signal.

10. The massage device of claim 1, wherein the heart rate detection sensor comprises a weight detection sensor configured to detect a weight of a body seated on the massage part.

11. The massage device of claim 1,
wherein the heart rate detection sensor is disposed on one side of the support part, and
wherein the massage part comprises a thermotherapeutic ceramic rotatably coupled to the support part and configured to provide thermotherapy to a user.

12. The massage device of claim 11,
wherein the thermotherapeutic ceramic and the heart rate detection sensor are each provided in plurality,
wherein the plurality of thermotherapeutic ceramics comprise a first thermotherapeutic ceramic and a second thermotherapeutic ceramic arranged along a first line parallel to a user's body length direction, and a third thermotherapeutic ceramic and a fourth thermotherapeutic ceramic arranged along a second line parallel to the first line, and
wherein the plurality of heart rate detection sensors comprise a first heart rate detection sensor disposed on the first line and a second heart rate detection sensor disposed on the second line.

13. A method for measuring a heart rate of a user receiving a massage from a massage device including a frame placed on a floor and a massage module installed on the frame to provide a massage, the method comprising:
obtaining a first signal from a heart rate detection sensor provided in the massage module;
obtaining a second signal from a vibration detection sensor provided on the frame; and
determining the user's heart rate based on the first signal and the second signal.

14. The method of claim 13,
wherein the first signal comprises a heart rate signal detected based on a user's heartbeat and a noise signal other than the heart rate signal, and
wherein the determining the user's heart rate comprises correcting the first signal based on the second signal so that the noise signal can be removed.

15. The method of claim 14,
wherein the correcting the first signal comprises:
determining a correction signal based on the second signal and a correction factor; and
determining the heart rate based on the first signal and the correction signal.

16. The method of claim 15,
wherein the correcting the first signal comprises:
adjusting the correction factor so that a noise signal of the first signal becomes identical to the correction signal.

17. The method of claim 16, wherein the adjusting the correction factor is performed using an adaptive filter algorithm.

18. The method of claim 13, further comprising amplifying and filtering at least one of the first signal and the second signal.
